SEQUENCE LISTING (24 sequences)

SEQ ID NO: 1 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 5 aa, Homo sapiens
SYTMH

SEQ ID NO: 4 - protein, 17 aa, Homo sapiens
FISYDGNNKYYADSVKG

SEQ ID NO: 5 - protein, 9 aa, Homo sapiens
TGWLGPFDY

SEQ ID NO: 6 - protein, 12 aa, Homo sapiens
RASQSVGSSYLA

SEQ ID NO: 7 - protein, 7 aa, Homo sapiens
GAFSRAT

SEQ ID NO: 8 - protein, 9 aa, Homo sapiens
QQYGSSPWT

SEQ ID NO: 9 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 10 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 11 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 447 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 10 aa, Homo sapiens
GFTFSSYGMH

SEQ ID NO: 15 - protein, 15 aa, Homo sapiens
VIWYDGSNKYYADSV

SEQ ID NO: 16 - protein, 16 aa, Homo sapiens
DPRGATLYYYYYGMDV

SEQ ID NO: 17 - protein, 11 aa, Homo sapiens
RASQSINSYLD

SEQ ID NO: 18 - protein, 7 aa, Homo sapiens
AASSLQS

SEQ ID NO: 19 - protein, 9 aa, Homo sapiens
QQYYSTPFT

SEQ ID NO: 20 - protein, 125 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 21 - protein, 107 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 22 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 450 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 24 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0002513 A1
(43) Pub. Date: Jan. 4, 2024

(54) DOSING AND ADMINISTRATION OF NON-FUCOSYLATED ANTI-CTLA-4 ANTIBODY AS MONOTHERAPY

(71) Applicant: BRISTOL-MYERS SQUIBB COMPANY, Princeton, NJ (US)

(72) Inventors: Leonard P. JAMES, Princeton, NJ (US); Yougan CHENG, Princeton, NJ (US); Brian J. SCHMDIT, Maple Glen, PA (US); John J. ENGELHARDT, Fremont, CA (US); Li LI, Skillman, NJ (US)

(21) Appl. No.: 18/251,935

(22) PCT Filed: Nov. 5, 2021
(86) PCT No.: PCT/US2021/058184

§ 371 Date: May 5, 2023

Related U.S. Application Data
(60) Provisional application No. 63/110,534, filed on Nov. 6, 2020.

(51) Int. Cl. C07K 16/28

(52) U.S. Cl. CPC C07K 16/2818; C07K 2317/41; A61K 2039/545

(57) ABSTRACT

The present invention provides methods of dosing and administration of non-fucosylated anti-CTLA-4 antibodies, such as non-fucosylated ipilimumab, as monotherapy, and related compositions and dosage forms.

### CROSS-REFERENCE TO RELATED APPLICATIONS

This application claims the benefit under 35 U.S.C. § 119(e) of U.S. Provisional Application Ser. No. 63/110,534, filed Nov. 6, 2020; the disclosure of which is incorporated herein by reference.

### SEQUENCE LISTING

The Sequence Listing filed electronically herewith is also hereby incorporated by reference in its entirety (File Name: 20211003_SEQL_13579WOPCT_GB.txt; Date Created: 3 Nov. 2021; File Size: 29 KB).

### FIELD OF THE INVENTION

The present application discloses methods of dosing and administration of non-fucosylated anti-CTLA-4 antibodies as monotherapy for treating cancer.

### BACKGROUND OF THE INVENTION

The immune system is capable of controlling tumor development and mediating tumor regression. This requires the generation and activation of tumor antigen—specific T cells. Multiple T-cell co-stimulatory receptors and T-cell negative regulators, or co-inhibitory receptors, act in concert to control T-cell activation, proliferation, and gain or loss of effector function. Among the earliest and best characterized T-cell co-stimulatory and co-inhibitory molecules are CD28 and CTLA-4. Rudd et al. (2009) Immunol. 229: 12. CD28 provides co-stimulatory signals to T-cell receptor engagement by binding to B7-1 and B7-2 ligands on antigen-presenting cells, while CTLA-4 provides a negative signal down-regulating T-cell proliferation and function. CTLA-4, which also binds the B7-1 (CD80) and B7-2 (CD86) ligands but with higher affinity than CD28, acts as a negative regulator of T-cell function through both cell autonomous (or intrinsic) and cell non-autonomous (or extrinsic) pathways. Intrinsic control of CD8⁺ and CD4 T effector (T_{eff}) function is mediated by the inducible surface expression of CTLA-4 as a result of T-cell activation, and inhibition of T-cell proliferation and cytokine proliferation by multivalent engagement of B7 ligands on opposing cells. (2008) Immunol. 224:141.

Anti-CTLA-4 antibodies, when cross-linked, suppress T cell function in vitro. Krummel & Allison (1995) J. 182:459; Walunas et al. (1994) Immunity 1:405. Regulatory T cells (T_{regs}), which express CTLA-4 constitutively, control T_{eff} function in a non-cell autonomous fashion. T_{regs} that are deficient for CTLA-4 have impaired suppressive ability (Wing et al. (2008) Science 322:271) and antibodies that block CTLA-4 interaction with B7 can inhibit T_{reg} function (Read et al. 192:295; Quezada et al. (2006) J. Clin. Invest. 116:1935). More recently, T_{effs} have also been shown to control T cell function through extrinsic pathways (Corse & Allison (2012) J. Immunol. 189:1123; Wang et al. (2012) J. Immunol. 189:1118). Extrinsic control of T cell function by T_{regs} and T_{effs} occurs through the ability of CTLA-4-positive cells to remove B7 ligands on antigen-presenting cells, thereby limiting their co-stimulatory potential. Qureshi et al. (2011) Science 332: 600; Onishi et al. (2008) (USA) 105:10113. Antibody blockade of CTLA-4/B7 interactions is thought to promote T_{eff} activation by interfering with negative signals transmitted by CTLA-4 engagement; this intrinsic control of T-cell activation and proliferation can promote both T_{eff} and T_{reg} proliferation (Krummel & Allison (1995) J. Med. 182:459; Quezada et al. (2006) J. Clin. Invest. 116:1935). In early studies with animal models, antibody blockade of CTLA-4 was shown to exacerbate autoimmunity. Perrin et al. (1996) J. 157:1333; Hurwitz et al. (1997)J. Neuroimmunot 73:57. By extension to tumor immunity, the ability of anti-CTLA-4 to cause regression of established tumors provided a dramatic example of the therapeutic potential of CTLA-4 blockade. Leach et al. (1996) Science 271:1734.

Human antibodies to human CTLA-4, ipilimumab and tremelimumab, were selected to inhibit CTLA-4-B7 interactions (Keler et al. (2003) J. Immunol 171:6251; Ribas et al. (2007) Oncologist 12:873) and have been tested in a variety of clinical trials for multiple malignancies. Hoos et al. (2010) Semin. Oncol. 37:533; Ascierto et al. (2011) J. Transl. Med. 9:196. Ipilimumab, which was first approved for the treatment of metastatic melanoma, has since been approved for use in other cancers, and is in clinical testing in yet other cancers. Hoos et al. (2010) Semin. Oncol. 37:533; Hodi et al. (2010) N. Engl. J. Med. 363:711; Pardoll (2012) Nat. Immunol. 13(12): 1129. In 2011, ipilimumab, which has an IgG1 constant region, was approved in the US and EU for the treatment of unresectable or metastatic melanoma based on an improvement in overall survival in a phase III trial of previously treated patients with advanced melanoma. Hodi et al. (2010) N. Engl. Med. 363:711. Tumor regressions and disease stabilization were frequently observed, but treatment with these antibodies has been accompanied by adverse events with inflammatory infiltrates capable of affecting a variety of organ systems.

Anti-CTLA-4 antibodies with enhanced antibody dependent cellular cytotoxicity (ADCC) activity, such as non-fucosylated anti-CTLA-4 antibodies, have been proposed as therapeutic agents for treatment of cancer through depletion of T_{regs}. Int'l Pat. App. Pub. No. WO 14/089113. The enhanced ADCC activity introduced by nonfucosylation of anti-CTLA-4 antibodies, however, may also deplete other CTLA-4 expressing cells, such as anti-tumor CD8⁺ T cells. The need exists for methods of dosing and administration of non-fucosylated anti-CTLA-4 antibodies, such as non-fucosylated ipilimumab, that maximize anti-tumor activity.

### SUMMARY OF THE INVENTION

The present invention provides methods of treatment of cancer with a non-fucosylated anti-CTLA-4 antibody in which the antibody is administered as monotherapy once every two weeks (Q2W) or once every four weeks (Q4W) at a flat does of 4 mg, 5 mg, 6 mg, 7 mg, 10 mg, 20 mg, 40 mg, 70 mg, 100 mg or 200 mg. In some embodiments, administration is Q2W and the dose is 4 mg, 5 mg, 6 mg, 7 mg or 10 mg. In other embodiments, administration is Q4W and the dose is 20 mg, 40 mg, 70 mg, 100 mg or 200 mg.

In one embodiment, the non-fucosylated anti-CTLA-4 antibody comprises the CDRH1, CDRH2, CDRH3, CDRL1, CDRL2 and CDRL3 sequences of SEQ ID NOs: 3-8, respectively. In another embodiment, the non-fucosylated anti-CTLA-4 antibody with enhanced ADCC activity comprises the V_{H} and V_{L} sequences of SEQ ID NOs: 9 and 10, respectively. In a further embodiment, the non-fucosylated anti-CTLA-4 antibody is ipilimumab comprising the HC sequence of SEQ ID NO: 11 or 12, and the LC sequence of SEQ ID NO: 13.

In an alternative embodiment, the non-fucosylated anti-CTLA-4 antibody comprises the CDRH1, CDRH2, CDRH3, CDRL1, CDRL2 and CDRL3 sequences of SEQ ID NOs: 14-19, respectively. In another embodiment, the non-fucosylated anti-CTLA-4 antibody comprises the V_{H} and V_{L} sequences of SEQ ID NOs: 20 and 21, respectively. In a further embodiment, the non-fucosylated anti-CTLA-4 antibody is tremelimumab comprising the HC sequence of SEQ ID NO: 22 or 23, and the LC sequence of SEQ ID NO: 24.

In various embodiments the methods of treating cancer of the present invention outlined above are used to treat a cancer selected from the group consisting of those in which T_{reg} biology may play an important role in cancer growth. These include but are not limited to: non-small cell lung cancer (NSCLC) (squamous and non-squamous), gastric cancer, triple-negative breast cancer (TNBC), colorectal cancer (CRC), squamous cell carcinoma of the head and neck (SCCHN), pancreatic cancer, metastatic castration resistant prostate cancer (mCRPC), and transitional cell cancer (urinary bladder) (TCC). In some such embodiments non-fucosylated anti-CTLA-4 antibody, such as non-fucosylated ipilimumab, is administered at a fixed dose of 7 mg, 20 mg, 70 mg or 100 mg.

In another embodiment the methods of treating cancer of the present invention outlined above are used to treat melanoma patients after failure of treatment with anti-PD-1 or anti-PD-L1 antibodies, which patients are referred to herein as “PD(L)1-progressed melanoma” patients. In some such embodiments non-fucosylated anti-CTLA-4 antibody, such as non-fucosylated ipilimumab, is administered at a fixed dose of 4 mg, 5 mg, 6 mg, 7 mg, 10 mg or 20 mg. In various embodiments, monotherapy is begun two to six weeks after the last dose of anti-PD-1 or anti-PD-L1 antibodies in the prior round of therapy, e.g. two weeks. In select embodiments, 5 mg to 7 mg is administered Q2W, or 20 mg is administered Q6W.

In another aspect, the invention provides use of a non-fucosylated anti-CTLA-4 antibody, such as non-fucosylated ipilimumab, in the manufacture of a medicament for treating cancer at a fixed dose selected from the group consisting of 4 mg, 5 mg, 6 mg, 7 mg, 10 mg, 20 mg, 40 mg, 70 mg, 100 mg and 200 mg. In some embodiments the medicament is provided in unit dose form, e.g. vials, prefilled syringes and autoinjectors; and/or the medicament is provided with instructions for administration of a fixed dose selected from the group consisting of 4 mg, 5 mg, 6 mg, 7 mg, 10 mg, 20 mg, 40 mg, 70 mg, 100 mg and 200 mg.

In a further aspect, the invention provides unit doses of a non-fucosylated anti-CTLA-4 antibody, wherein the unit dose is selected from the group consisting of 4 mg, 5 mg, 6 mg, 7 mg, 10 mg, 20 mg, 40 mg, 70 mg, 100 mg and 200 mg. In various embodiments, the unit doses of the present invention are provided in vials, prefilled syringes and autoinjectors.

### BRIEF DESCRIPTION OF THE DRAWINGS

FIGS. 1A-1D show the results of clinical trial results for virtual patients in a virtual clinical trial to determine optimum dosing of non-fucosylated ipilimumab in nivolumab-progressed melanoma patients. See Example 2. FIGS. 1A, 1B, 1C and 1D provide complete response (CR), partial response (PR), stable disease (SD) and progressive disease (PD) for various doses of ipilimumab-NF, and for 3 mg/kg ipilimumab. All dosing was Q4W except ipilimumab, which was dosed Q3W, and all dosing began two weeks after the last dose of nivolumab. Values were averaged from 100 virtual trials where each trial had 100 virtual patients.

FIGS. 2A and 2B show results analogous to those in FIGS. 1A-1D using a refined, second iteration, QSP model, as described in Example 2. FIGS. 2A and 2B provide response rates for various doses of ipilimumab-NF administered Q4W, and 3 mg/kg ipilimumab dosed Q3W, starting two weeks or six weeks after the last dose of nivolumab, respectively. Values are averaged from 100 virtual trials where each trial has 100 virtual patients.

FIG. 3 provides additional results analogous to those in FIGS. 2A and 2B, with the listed doses of non-fucosylated anti-CTLA-4 antibody administered Q4W starting two weeks after the last nivolumab dose.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Administering,” “administer” or “administration” refers to the physical introduction of a composition comprising a therapeutic agent to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

Unless otherwise indicated, administration of antibodies for the treatment of cancer is parenteral, such as intravenous (iv) or subcutaneous (sc). Methods of dosing and administration of the present invention can be performed for any number of cycles of treatment, from one, two, three, four cycles, etc., up to continuous treatment (repeating the dosing until no longer necessary, disease recurrence, or unacceptable toxicity is reached). For the purposes of the present disclosure, one cycle comprises the minimal unit of administration that includes one dose of the therapeutic agent.

“Initial Dose” or “initial dosing” as used herein refers to the first dosing of a patient with the regimen, and any subsequent repetitions of that same dosing regimen (such as second, third and fourth cycles, etc.), and is contrasted with “maintenance dose” or “maintenance dosing,” which refers to subsequent doses administered over a longer period after the initial dose or doses, e.g. longer than three months up to several years, or even indefinitely. Maintenance dosing may optionally comprise less frequent dosing and/or lower dose than the initial dose, but in some cases, e.g. following a previous round of treatment with an earlier different drug, the initial dose may be lower than subsequent maintenance doses, e.g. due to combination effects with residual levels of the earlier drug that are higher during the initial dose than with subsequent maintenance doses.

An “antibody” (Ab) shall include, without limitation, a glycoprotein immunoglobulin which binds specifically to an antigen and comprises at least two heavy chains (HC) and two light chains (LC) interconnected by disulfide bonds. Each heavy chain comprises a heavy chain variable region (abbreviated herein as V_{H}) and a heavy chain constant region. The heavy chain constant region comprises three domains, C_{H1}, C_{H2} and CH₃. Each light chain comprises a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Antibody chains may be substantially identical but not entirely identical if they differ due to post-translational modifications, such as C-terminal cleavage of lysine residues, alternative glycosylation patterns, etc. Antibodies differing in fucosylation within the glycan, however, are not substantially identical.

Unless indicated otherwise or clear from the context, an antibody defined by its target specificity (e.g. an “anti-CTLA-4 antibody”) refers to antibodies that can bind to its human target (i.e. human CTLA-4). Such antibodies may or may not bind to CTLA-4 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. “Isotype” refers to the antibody class (e.g., IgM or IgG1) that is encoded by the heavy chain constant region genes. “Antibody” includes, by way of example, both naturally occurring and non-naturally occurring antibodies, including allotypic variants; monoclonal and polyclonal antibodies; chimeric and humanized antibodies; human or non-human antibodies; wholly synthetic antibodies; and single chain antibodies. Unless otherwise indicated, or clear from the context, antibodies disclosed herein are human IgG1 antibodies.

The term “monoclonal antibody” (“mAb”) refers to a preparation of antibody molecules of single molecular composition, i.e., antibody molecules whose primary sequences are essentially identical, and which exhibit a single binding specificity and affinity for a particular epitope. Monoclonal antibodies may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously herein.

A “humanized” antibody refers to an antibody having CDR regions derived from non-human animal, e.g. rodent, immunoglobulin germ line sequences in which some, most or all of the amino acids outside the CDR domains are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “antibody fragment” refers to a portion of a whole antibody, generally including the “antigen-binding portion” (“antigen-binding fragment”) of an intact antibody which retains the ability to bind specifically to the antigen bound by the intact antibody.

“Antibody-dependent cell-mediated cytotoxicity” (ADCC) refers to an in vitro or in vivo cell-mediated reaction in which nonspecific cytotoxic cells that express FcRs (e.g., natural killer (NK) cells, macrophages, neutrophils and eosinophils) recognize antibody bound to a surface antigen on a target cell and subsequently cause lysis of the target cell. In principle, any effector cell with an activating FcR can be triggered to mediate ADCC. Unless otherwise indicated, ADCC can be measured by an assay substantially similar to the assay provided in Example 1.

“Unit dose” as used herein refers to a single sterile package of drug, such as non-fucosylated anti-CTLA-4 antibody of the present invention, wherein the amount of drug provided is equivalent to a prescribed fixed dose of the drug. Unless otherwise indicated, a unit dose is defined by the nominal amount of drug present which equals the prescribed dose, and does not include any overfill. The nominal dose refers to the amount of drug prescribed for the patient, i.e. the intended amount to be administered to the patient.

“Overfill” refers to additional drug (and related other components) provided in a unit dose above and beyond the nominal dose. Unless otherwise indicated, the amount of drug in a given unit dose includes sufficient overfill, e.g. 0.7 ml, to allow for safe and convenient withdrawal of the full nominal volume of drug solution, and thus the complete dose, e.g. without getting air in the hypodermic needle used to withdraw the sample for injection.

“Cancer” refers a broad group of various diseases characterized by the uncontrolled growth of abnormal cells in the body. Unregulated cell division and growth divide and grow results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

“Effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and down-regulation of a cell surface receptor (e.g., the B cell receptor; BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) receptors and one inhibitory (FcγRIIB) receptor. Various properties of human FcγRs are summarized in Table 1. The majority of innate effector cell types co-express one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans.

TABLE 1
Properties of Human FcγRs

| | Allelic | Affinity for | Isotype | |
|---|---|---|---|---|
| Fcγ | variants | human IgG | preference | Cellular distribution |
| FcγRI | None | High | IgG1 = 3 > 4 >> 2 | Monocytes, macrophages, |
| | described | (K_{D}~10 nM) | | activated neutrophils, |
| | | | | dendritic cells? |
| FcγRIIA | H131 | Low to | IgG1 > 3 > 2 > 4 | Neutrophils, monocytes, |
| | | medium | | macrophages, eosinophils, |
| | R131 | Low | IgG1 > 3 > 4 > 2 | dendritic cells, platelets |
| FcγRIIIA | V158 | Medium | IgG1 = 3 >> 4 > 2 | NK cells, monocytes, |
| | F158 | Low | IgG1 = 3 >> 4 > 2 | macrophages, mast cells, |
| | | | | eosinophils, dendritic cells? |
| FcγRIIB | I232 | Low | IgG1 = 3 = 4 > 2 | B cells, monocytes, |
| | T232 | Low | IgG1 = 3 = 4 > 2 | macrophages, dendritic |
| | | | | cells, mast cells |

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (Clq) of the classical complement system. Thus, the Fc region is a polypeptide comprising the constant region of an antibody excluding the first constant region immunoglobulin domain. In IgG, IgA and IgD antibody isotypes, the Fc region is composed of two identical protein fragments, derived from the second (C_{H2}) and third (C_{H3}) constant domains of the antibody's two heavy chains; IgM and IgE Fc regions contain three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc or a variant Fc. Fc may also refer to this region in isolation or in the context of an Fc-comprising protein polypeptide such as a “binding protein comprising an Fc region,” also referred to as an “Fc fusion protein” (e.g., an antibody or immunoadhesin).

“Fucosylation,” and “non-fucosylation” or synonymously “afucosylated,” as used herein, refer to the presence or absence of a core fucose residue on the N-linked glycan at position N297 of an antibody (EU numbering).

“Non-fucosylated ipilimumab,” as used herein, refers to ipilimumab in which N-linked glycan comprises a core fucose residue in 5% or less of antibody heavy chains, including 2% or less, 1% or less and 0%. Ipilimumab, as contrasted with non-fucosylated ipilimumab, carries normal levels of fucosylation found in antibodies produced in CHO cells with a competent α-1,6 fucosylation pathway, e.g. the level of core fucosylation on N-linked glycans found in YERVOY®, such as 98 to 99%, or at least 95%. Unless otherwise indicated, “ipilimumab” refers to the form of the antibody with normal levels of fucosylation, and is to be distinguished from “non-fucosylated ipilimumab.”

An “immune response” refers to a biological response within a vertebrate against foreign agents, which response protects the organism against these agents and diseases caused by them. The immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response. “Immuno-oncology” refers to treatment of cancer using one or more agents that enhance anti-tumor immune response.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeably herein with “polypeptide.”

A “subject” includes any human or non-human animal. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments of any aspect of the disclosed invention, the subject is a human. Unless otherwise indicated, a subject as referred to herein is a human. The terms “subject” and “patient” are used interchangeably herein.

A “therapeutically effective amount” or “therapeutically effective dosage” of a drug or therapeutic agent, such as an Fc fusion protein of the invention, is any amount of the drug that promotes disease regression evidenced by a decrease in severity of disease symptoms, an increase in frequency and duration of disease symptom-free periods, or a prevention of impairment or disability due to the disease affliction. Effectiveness is measured with reference to the natural course of disease in the absence of treatment, and thus includes treatment that slows disease progression. A “prophylactically effective amount” or “prophylactically effective dosage” refers to any amount of the drug that, when administered to a subject at risk of developing a disease or of suffering a recurrence of disease, inhibits the development or recurrence of the disease. The ability of a therapeutic agent to promote disease regression, or a prophylactic agent to inhibit, the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent promotes cancer regression in a subject, or prevents or limits disease progression that would otherwise occur in the absence of treatment. In preferred embodiments, a therapeutically effective amount of the drug promotes cancer regression to the point of eliminating the cancer. “Promoting cancer regression” means that administering an effective amount of the drug results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. In other examples an anti-cancer agent may slow disease progression or cause stable disease in a subject that would otherwise have experienced progressive disease. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated in an animal model system, such as the CT26 colon adenocarcinoma, MC38 colon adenocarcinoma and SalN fibrosarcoma mouse tumor models, which are predictive of efficacy in human tumors. Alternatively, this property of a composition can be evaluated by examining the ability of the compound to inhibit cell growth, such inhibition can be measured in vitro by assays known to the skilled practitioner. In other preferred embodiments of the invention, tumor regression may be observed and continue for a period of at least about 20 days, more preferably at least about 40 days, or even more preferably at least about 60 days.

“Treatment” or “therapy” of a subject refers to any type of intervention or process performed on, or administering an active agent to, the subject with the objective of reversing, alleviating, ameliorating, inhibiting, slowing down or prevent the onset, progression, development, severity or recurrence of a symptom, complication, condition or biochemical indicia associated with a disease.

“Monotherapy,” as used herein, refers to treatment of a human subject with an anti-CTLA-4 antibody with enhanced ADCC, such as a non-fucosylated anti-CTLA-4 antibody, including but not limited to non-fucosylated ipilimumab (BMS-986218), in the absence of concurrent treatment with any other immunotherapy agent or agents. Concurrent treatment refers to coordinated dosing and administration of one or more additional immunotherapy agents, including but not limited to an anti-PD-1 and/or anti-PD-L1 antibody, in a single treatment regimen in which doses of the additional immunotherapy agent(s) are administered at the same time, including as a co-formulated composition, or are administered at overlapping or staggered intervals, with the anti-CTLA-4 antibody with enhanced ADCC, in one or more treatment cycles. Monotherapy does not preclude concurrent treatment with non-immunotherapy therapeutic agents, including but not limited to drugs to treat side effects of immunotherapy. Monotherapy also does not preclude prior therapy with immunotherapeutic agent(s) as part of a separate therapeutic regimen, for example when monotherapy is used as second (or later) line therapy. Monotherapy further does not preclude subsequent treatment with immunotherapeutic agent(s) as part of a separate therapeutic regimen after monotherapy. In one embodiment. administration of the anti-CTLA-4 antibody with enhanced ADCC according to the present invention may begin following a treatment regimen with a different immunotherapy agent, e.g. within as little as a week or two, and still comprise monotherapy if the one or more rounds of treatment with the anti-CTLA-4 antibody with enhanced ADCC according to the present invention are not concurrent with, or interspersed with, doses of such different immunotherapy agent.

“Anti-PD-1 antibody,” as used herein, includes any approved (by any health authority) therapeutic antibody that binds to human PD-1, including but not limited to, nivolumab, pembrolizumab, cemiplimab, and dostarlimab.

“Anti-PD-L1 antibody,” as used herein, includes any approved (by any health authority) therapeutic antibody that binds to human PD-L1, including but not limited to, atezolizumab, avelumab, and durvalumab.

### Dosing of Anti-CTLA-4 Antibodies

It is now recognized that CTLA-4 exerts its physiological function primarily through two distinct effects on the two major subsets of CD4⁺ T cells: (1) down-modulation of helper T cell activity, and (2) enhancement of the immunosuppressive activity of regulatory T cells (T_{regs}). Lenschow et al. (1996) Ann. Rev. Immunol. 14:233; Wing et al. (2008) Science 322:271; Peggs et al. (2009) J. Exp. Med. 206:1717. T_{regs} are known to constitutively express high levels of surface CTLA-4, and it has been suggested that this molecule is integral to their regulatory function. Takahashi et al. (2000) J. Exp. Med. 192:303; Birebent et al. (2004) Eur. J. Immunol. 34:3485. Accordingly, the T_{reg} population may be most susceptible to the effects of CTLA-4 blockade. Studies of ipilimumab patients also show that responders, as distinguished from non-responders, exhibit decreased T_{reg} infiltration after treatment, with depletion occurring via an ADCC mechanism and mediated by FcγRIIIA-expressing non-classical (CD14⁺CD16⁺⁺) monocytes. Romano et al. (2014) J. Immunotherapy of Cancer 2(Suppl. 3):014.

The only approved anti-CTLA-4 antibody, ipilimumab (YERVOY®), provides long-term survival in up to 25% of metastatic melanoma patients when administered at 3 mg/kg (metastatic melanoma) or 10 mg/kg (adjuvant melanoma), but treatment is often accompanied by toxicity. These doses correspond to fixed doses of approximately 240 mg and 800 mg, respectively (80 kg/patient). More specifically, for metastatic or unresectable melanoma, YERVOY® is administered intravenously at 3 mg/kg over 90 minutes every three weeks (Q3W) for a total of four doses. For adjuvant use in melanoma, YERVOY® is administered intravenously at 10 mg/kg over 90 minutes Q3W for a total of four doses, and every 12 weeks (Q12W) thereafter up to three years. For use in combination with anti-PD-1 antibody OPDIVO® (nivolumab) for hepatocellular carcinoma, YERVOY® is administered intravenously at 3 mg/kg Q3W for a total of four doses. For use in combination with anti-PD-1 antibody OPDIVO® (nivolumab) for advanced renal cell carcinoma or microsatellite instability-high (MSI-H) or mismatch repair deficient (dMMR) metastatic colorectal cancer, YERVOY® is administered intravenously at 1 mg/kg over 30 minutes Q3W for a total of four doses. Ipilimumab has a half-life of 15.4 days. YERVOY® Prescribing Information, updated March 2020.

### Non-Fucosylated Anti-CTLA-4 Antibodies

Tumors can evade immunosurveillance by both suppressing anti-tumor response and activating immunosuppressive pathways. The tumor microenvironment (TME) is frequently enriched for regulatory T cells (T_{regs}), helping explain the immunosuppressive environment of the TME. Treatment of cancer with anti-CTLA-4 antibodies, such as ipilimumab, expands CD8⁺ T cells, including anti-tumor CD8⁺ T cells, in lymphoid tissues by blocking the inhibitory signals that would otherwise result from the interaction of CTLA-4 with B7-1 and B7-2. Non-fucosylated ipilimumab has enhanced affinity for human activating Fcγ receptors, such as CD16/FcγRIII, on NK cells and macrophages, resulting in enhanced ADCC-mediated T_{reg} lysis activity compared with ipilimumab. Engelhardt et al. (2020) American Association for Cancer Research (AACR) Meeting, Poster 4552; see also commonly-assigned See also Int'l Pat. App. Pub. No. WO 18/160536 at FIG. 10. A non-fucosylated, enhanced ADCC anti-CTLA-4 antibody shows greater activity in an MC38 tumor model than a normally fucosylated anti-CTLA-4 mAb. Id.

Non-fucosylated ipilimumab (BMS-986218) has entered a phase 1/2a clinical trial in patients with advanced solid tumors. Clinical Trials.gov Identifier NCT03110107 (first posted 12 Apr. 2017). BMS-986218 was administered intravenously (IV) to patients with one or more prior therapy at 2 mg to 70 mg every four weeks. Treatment-related adverse events (TRAEs) occurred in 52% of monotherapy patients, but only 12% were grade 3, there were no grade 4 TRAEs, and a single Grade 5 (pneumonitis at 2 mg dose). The half-life of BMS-986218 was approximately two weeks, much like ipilimumab. Increased levels of serum chemokine ligands 9 (CXCL9) and 10 (CXCL10), and interferon-γ (IFN-γ) show that pharmacological changes induced by 2 mg of BMS-986218 (˜0.03 mg/kg) are similar to those induced by ipilimumab at 3 mg/kg, and changes induced by 40-70 mg of BMS-986218 (˜0.6-1 mg/kg) (67 kg/patient) are similar to those induced by ipilimumab at 10 mg/kg. In a subset of patients with paired biopsies, treatment with BMS-986218 was associated with an increased gene signature linked to CD8⁺ T-cell infiltration and inflammation.

BMS-986218 has also entered a clinical trial in combination with degarelix for prostate cancer, in which BMS-986218 is administered intravenously (IV) at 20 mg every two weeks for two doses starting three weeks prior to radical prostatectomy, plus degarelix 240 mg subcutaneous (SQ) for one dose two weeks prior to radical prostatectomy. Clinical Trials.gov Identifier NCT04301414 (first posted 10 Mar. 2020).

### Dosing of Non-Fucosylated Anti-CTLA-4 Antibodies

Without intending to be limited by theory, treatment of cancer with non-fucosylated anti-CTLA-4 antibodies of the present invention, such as non-fucosylated ipilimumab, enhances ADCC-mediated depletion of T_{regs} in the TME, enhancing the therapeutic mechanism, while retaining the benefits of CTLA-4 blockade in lymphoid tissues.

The increase in ADCC activity of non-fucosylated anti-CTLA-4 antibody, however, would be expected to deplete all CTLA-4 expressing cells in the TME, not just T_{regs}, including CTLA-4 expressing CD8⁺ T cells. The potential for depletion of both immunosuppressive T_{regs}, necessary to reduce the immunosuppressive environment in the TME, and also the cytotoxic anti-tumor CD8⁺ T cells necessary to eradicate tumors, makes dose selection critical. Optimal efficacy in the light of these conflicting mechanisms requires finding a dose that is effective in depleting T_{regs} in the TME while leaving sufficient anti-tumor CD8⁺ T cells to effect tumor eradication.

In one aspect, the present invention provides improved methods of dosing and administration of non-fucosylated anti-CTLA-4 antibodies, such as non-fucosylated ipilimumab at Q2W or Q4W dosing. Without intending to be limited by theory, dosing at Q2W is intended to reduce the recovery of intratumoral T_{reg} populations between doses, which might otherwise provide enough transient immunosuppression in the TME to negate anti-tumor immune response. The enhanced ADCC activity afforded by non-fucosylation of ipilimumab is expected to enhance T_{reg} depletion in the TME, thus enhancing anti-tumor efficacy, but it may also increase peripheral immune-mediated toxicity due to T_{reg} depletion in the periphery. Based in part on in vitro experiments showing ten-fold higher ADCC for non-fucosylated ipilimumab, and based in part on results from early stage human clinical trials, optimal dosing of non-fucosylated ipilimumab is lower than the dosing for ipilimumab, and includes, but is not limited to, fixed doses of 20 mg, 40 mg, 70 mg, 100 mg and 200 mg, dosed Q2W or Q4W.

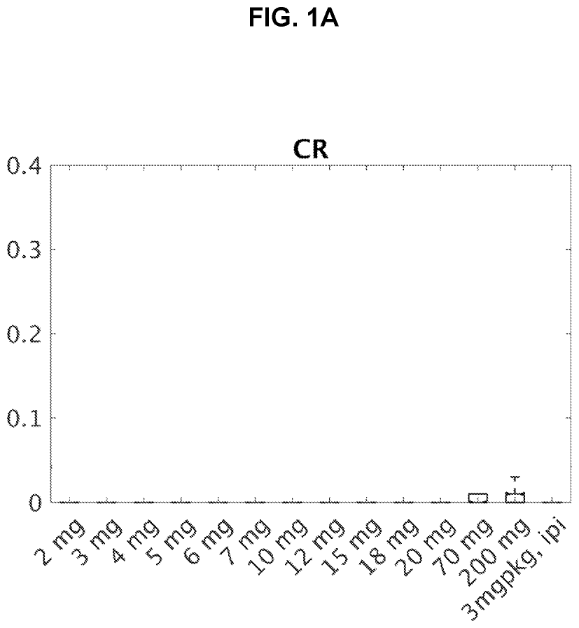

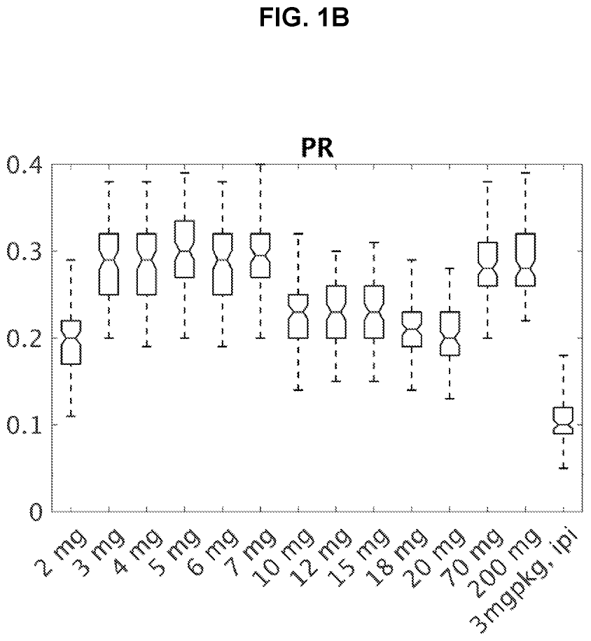

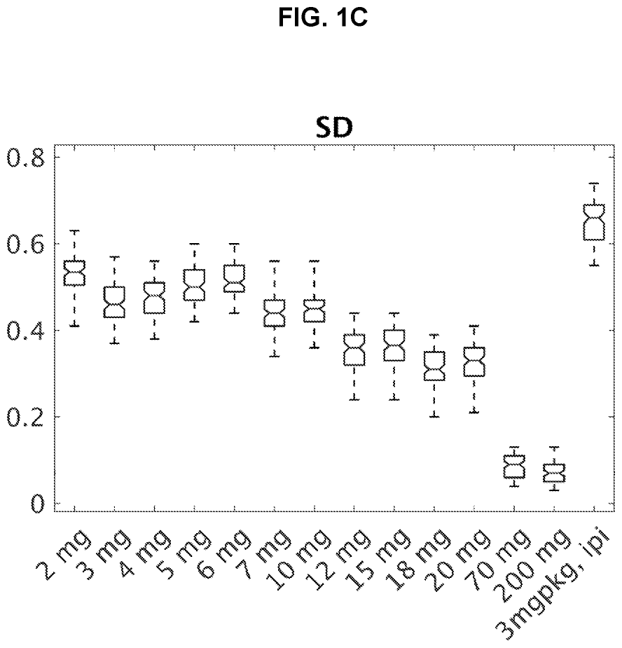

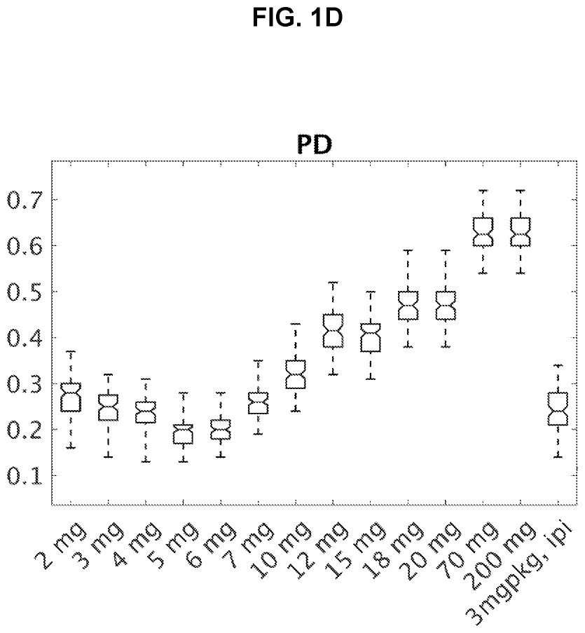

In addition, pharmacological modeling suggests that even lower doses may be optimal, e.g. in treatment of PD(L)1-progressed melanoma patients. See Example 2 and FIGS. 1A-1D, FIGS. 2A-2B, FIG. 3. Based on these modeling data, Applicants have surprisingly found that doses of non-fucosylated ipilimumab between 4 mg and 10 mg, such as 5 mg, 7 mg or 10 mg, dosed Q2W or Q4W, are optimally effective in treatment of PD(L)1-progressed melanoma when administered two weeks after the last dose of nivolumab. Interestingly, the optimum dose increases to 20 mg when non-fucosylated ipilimumab is administered six weeks after the last dose of nivolumab, i.e. after a six week washout period. See Example 2. This result shows the power of the model to account for the effects of residual nivolumab and/or physiological changes within the tumor over time on the efficacy of treatment with non-fucosylated ipilimumab, and specifically on the dose needed for optimal anti-tumor response.

Therapeutic efficacy of anti-tumor agents often improves continuously with increasing dose, and is limited only by toxicity. The low doses of non-fucosylated anti-CTLA-4 antibodies for the treatment of PD(L)1-progressed melanoma provided in the present invention are well below toxic levels. Rather than being limited by toxicity, the efficacious doses of the present invention are limited by a complex interplay of multiple competing effects of treatment on various cell types in various compartments, as represented in the quantitative systems pharmacology (QSP) model outlined in Example 2. The dose suggested by the QSP model is far lower than the approved doses for ipilimumab monotherapy, which are 3 mg/kg and 10 mg/kg Q3W for melanoma and adjuvant melanoma respectively, corresponding to approximately 240 mg and 800 mg fixed doses. One of skill in the art would recognize that precise dosing within the range from 4 mg to 10 mg may comprise any value within that range, including but not limited to integral values of 4 mg, 5 mg, 6 mg, 7 mg, 8 mg, 9 mg and 10 mg. Although such low doses were found in models of treatment of PD(L)-1 progressed melanoma patients, the same complex interplay of biological effects may occur in cancers more generally, making such low doses useful in the treatment of a wide range of cancers. In some embodiments the non-fucosylated anti-CTLA-4 antibody used in low dose methods of treating cancer of the present invention is non-fucosylated ipilimumab.

Dosing and administration for monotherapy with an anti-CTLA-4 antibody with enhanced ADCC activity, such as non-fucosylated ipilimumab, cannot be deduced from dosing and administration of approved anti-CTLA-4 antibodies like ipilimumab due to critical differences in their mechanisms of action. Dosing and administration for monotherapy with an immunotherapy agent may also differ substantially from dosing and administration of the same agent in combination therapy with one or more other immunotherapy agents, since the combined effects of multiple immunotherapy agents, especially those acting by a different mechanism, are unpredictable. Unexpected results may be observed on both therapeutic efficacy and side effects, which are typically dose-limiting in immunotherapy. Combinations of three or more agents are even less predictable than pairwise combinations.

### Unit Dose Formulations of Non-Fucosylated Anti-CTLA-4 ANTIBODIES

Further provided are compositions, e.g., pharmaceutical compositions, containing fixed doses of non-fucosylated anti-CTLA-4 antibodies formulated together with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

A composition described herein can be administered via one or more routes of administration using one or more of a variety of methods known in the art. As will be appreciated by the skilled artisan, the route and/or mode of administration will vary depending upon the desired results. Preferred routes of administration for antibodies described herein include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Non-fucosylated anti-CTLA-4 antibodies may be prepared as unit dose forms for administration according to the methods of the present invention. Such unit dose forms include single dose preparations comprising the requisite dose of non-fucosylated anti-CTLA-4 antibody, such as 4 mg, 5 mg, 7 mg, 10 mg, 40 mg, 20 mg, 70 mg, 100 mg or 200 mg, and a pharmaceutically acceptable carrier. Such unit dose forms may be contained in any appropriate vessel, including but not limited to a vial, a prefilled syringe or an autoinjector. Such unit dose forms may further comprise sufficient overfill to allow safe and convenient withdrawn of the nominal therapeutic dose from the vessel. In some embodiments the non-fucosylated anti-CTLA-4 antibody in unit dose form is non-fucosylated ipilimumab.

### Use of Non-Fucosylated Anti-CTLA-4 Antibodies in the Manufacture of a Medicament

In another aspect, the present invention provides use of non-fucosylated anti-CTLA-4 antibodies, such as non-fucosylated ipilimumab, in the manufacture of a medicament for administration at a fixed dose of 4 mg, 5 mg, 7 mg, 10 mg, 40 mg, 20 mg, 70 mg, 100 mg or 200 mg, optionally at one or more intervals of Q2W or Q4W. In one embodiment the medicament is for the treatment of cancer. Such medicaments may optionally be packaged in single dose units for convenience of administration and maintenance of sterility. Non-fucosylated anti-CTLA-4 medicaments of the present invention, whether provided as a single dose unit or otherwise, may optionally include instructions for use indicating administration at a fixed dose of 4 mg, 5 mg, 7 mg, 10 mg, 40 mg, 20 mg, 70 mg, 100 mg or 200 mg at intervals of Q2W or Q4W. In some embodiments the non-fucosylated anti-CTLA-4 antibody in the medicament is non-fucosylated ipilimumab.

### Reduced Fucosylation, Nonfucosylation and Hypofucosylation

The interaction of antibodies with FcγRs can be enhanced by modifying the glycan moiety attached to each Fc fragment at the N297 residue. In particular, the absence of core fucose residues strongly enhances ADCC via improved binding of IgG to activating FcγRIIIA without altering antigen binding or CDC. Natsume et al. (2009) Drug Des. Devel. Ther. 3:7. There is convincing evidence that non-fucosylated tumor-specific antibodies translate into enhanced therapeutic activity in mouse models in vivo. Nimmerjahn & Ravetch (2005) Science 310:1510; Mossner et al. (2010) Blood 115:4393.

Modification of antibody glycosylation can be accomplished by, for example, expressing the antibody in a host cell with altered glycosylation machinery. Antibodies with reduced or eliminated fucosylation, which exhibit enhanced ADCC, are particularly useful in the methods of the present invention. Cells with altered glycosylation machinery have been described in the art and can be used as host cells in which to express recombinant antibodies of this disclosure to thereby produce an antibody with altered glycosylation. For example, the cell lines Ms704, Ms705, and Ms709 lack the fucosyltransferase gene, FUT8 (α-(1,6) fucosyltransferase (see U.S. Pat. App. Publication No. 20040110704; Yamane-Ohnuki et al. (2004) Biotechnol. Bioeng. 87: 614), such that antibodies expressed in these cell lines lack fucose on their carbohydrates. As another example, EP 1176195 also describes a cell line with a functionally disrupted FUT8 gene as well as cell lines that have little or no activity for adding fucose to the N-acetylglucosamine that binds to the Fc region of the antibody, for example, the rat myeloma cell line YB2/0 (ATCC CRL 1662). PCT Publication WO 03/035835 describes a variant CHO cell line, Lec13, with reduced ability to attach fucose to Asn(297)-linked carbohydrates, also resulting in hypofucosylation of antibodies expressed in that host cell. See also Shields et al. (2002) J. Biol. Chem. 277:26733. Antibodies with a modified glycosylation profile can also be produced in chicken eggs, as described in PCT Publication No. WO 2006/089231. Alternatively, antibodies with a modified glycosylation profile can be produced in plant cells, such as Lemna. See e.g. U.S. Publication No. 2012/0276086. PCT Publication No. WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies. See also Umaña et al. (1999) Nat. Biotech. 17:176. Alternatively, the fucose residues of the antibody may be cleaved off using a fucosidase enzyme. For example, the enzyme alpha-L-fucosidase removes fucosyl residues from antibodies. Tarentino et al. (1975) Biochem. 14:5516. Antibodies with reduced fucosylation may also be produced in cells harboring a recombinant gene encoding an enzyme that uses GDP-6-deoxy-D-lyxo-4-hexylose as a substrate, such as GDP-6-deoxy-D-lyxo-4-hexylose reductase (RMD), as described at U.S. Pat. No. 8,642,292. Alternatively, cells may be grown in medium containing fucose analogs that block the addition of fucose residues to the N-linked glycan or a glycoprotein, such as antibody, produced by cells grown in the medium. U.S. Pat. No. 8,163,551; WO 09/135181.

In selected embodiments, non-fucosylated antibodies are produced in cells lacking an enzyme essential to fucosylation, such as FUT8 (e.g. U.S. Pat. No. 7,214,775), or in cells in which an exogenous enzyme partially depletes the pool of metabolic precursors for fucosylation (e.g. U.S. Pat. No. 8,642,292), or in cells cultured in the presence of a small molecule inhibitor of an enzyme involved in fucosylation (e.g. WO 09/135181).

In some embodiments the level of non-fucosylation is structurally defined. As used herein, non-fucosylated or afucosylated (terms used synonymously) antibody preparations are antibody preparations comprising greater than 95% non-fucosylated antibody heavy chains, including 100%.

The level of fucosylation in an antibody preparation may be determined by any method known in the art, including but not limited to gel electrophoresis, liquid chromatography, and mass spectrometry. Unless otherwise indicated, for the purposes of the present invention, the level of fucosylation in an antibody preparation is determined by hydrophilic interaction chromatography (or hydrophilic interaction liquid chromatography, HILIC), essentially as described at Example 3. To determine the level of fucosylation of an antibody preparation, samples are denatured and treated with PNGase F to cleave N-linked glycans, which are then analyzed for fucose content. LC/MS of full-length antibody chains is an alternative method to detect the level of fucosylation of an antibody preparation, but mass spectroscopy is inherently less quantitative.

The present invention is further illustrated by the following examples, which should not be construed as limiting. The contents of all figures and all references, patents and published patent applications cited throughout this application are expressly incorporated herein by reference.

### EXAMPLE 1

### Enhanced ADCC by Non-Fucosylated Ipilimumab as Measured by Promotion of NK-Mediated Cell Lysis Using Primary Human Cells

Non-fucosylated ipilimumab is tested for its ability to promote NK cell-mediated lysis of T_{regs} from a human donor as follows. Briefly, T_{regs} for use as target cells are separated by negative selection using magnetic beads and activated for 72 hours. NK cells for use as effectors from a human donor are separated by negative selection using magnetic beads and activated with IL-2 for 24 hrs. Calcein-labeled activated T_{regs} (Donor Leukopak AC8196) are coated with various concentrations of ipilimumab, ipilimumab-NF or an IgG1 control for 30 minutes, and then incubated with NK effector cells at a ratio of 10:1 for 2 hours. Calcein release is measured by reading the fluorescence intensity of the media using an Envision plate reader (Perkin Elmer), and the percentage of antibody-dependent cell lysis is calculated based on mean fluorescence intensity (MFI) with the following formula: [(test MFI−mean background)/(mean maximum−mean background)]×100.

### EXAMPLE 2

### Quantitative Systems Pharmacology (QSP) Model

The effects of non-fucosylated anti-CTLA-4 antibody on treatment of PD(L)1-progressed melanoma patients were assessed using a quantitative systems pharmacology (QSP) model. QSP models are described generally at Musante et al. (2017) “Quantitative Systems Pharmacology: A Case for Disease Models.” Clinical Pharmacology & Therapeutics 101:24. QSP models have a range of applications, including guiding dose and dose regimen optimization, characterizing pharmacodynamic biomarkers, assessing combination therapies, clinical trial power, and assessing candidate stratification markers for responding patients.

The QSP model used to support non-fucosylated anti-CTLA-4 antibody dosing simulations incorporated major components of cancer immunity cycle, including CTLA-4 and PD-1 pathways, immune cells such as CD8⁺ T cells and regulatory T cells, additional immune cells, cell-cell contact dynamics, cell life cycles and tissue recruitment, cytokine mediated feedback loops, ADCC, cancer killing and important clinically measured dynamics such as lesion response and immune cell counts. This combination therapeutic response QSP model was developed in SIMBIOLOGY® modeling and simulation software package (MathWorks Inc., Natick, Mass., USA). The non-fucosylated anti-CTLA-4 antibody in the model was based on non-fucosylated ipilimumab. At the start of the study (first iteration) the model initially included about 66 species, 236 reactions and more than 250 literature references, and many pathway model fits were used to determine model structures and parameters. In a second iteration of model development, the model was expanded to about 131 species, 370 reactions, and 398 rules.

After developing the model pathways, the next step was to ensure the model was able to capture and explain observed clinical trial biomarker and response variability. We applied a virtual population (VPop) calibration strategy for this. Cheng et al. (2020) “A virtual population (VPop) development workflow with improved efficiency and scale applied to immuno-oncology quantitative systems pharmacology models (I-O QSP platforms),” 11th American Conference on Pharmacometrics, ACoP11,Virtual, ISSN:2688-3953, at THU-093; and Cheng & Schmidt (2019) “An automated iterative virtual population development workflow for calibration of multi-therapy immuno-oncology quantitative systems pharmacology models (I-O QSP platforms) to population data from the clinical setting,” 10th American Conference on Pharmacometrics, ACoP10, Orlando FL, ISSN:2688-3953, at M-081.

Parameters from the QSP model were selected to vary to represent a virtual patient (VP). The following were considered when defining a VP: 1. characterized variation in pathway models, 2. observed literature discrepancies, and 3. the ability of the model to recreate variability in observed patient characteristics (e.g. immune cell content in blood, tumor, tumor draining lymph node). Then we generated a VP cohort with the QSP model using acceptance-rejection criteria to ensure each VP has reasonable phenotypes. The following steps were taken:
i) Alternate model parameterizations were sampled (biomarker and response diversity);
ii) Multiple interventions (therapies) were simulated for each VP;
iii) Clinical biomarker and response data were used to guide reasonable parameter bounds and set acceptance criteria on simulated outcomes; and
iv) Plausible VPs were required to pass acceptance criteria.

After developing a VP cohort, we then built a VPop that matched observed statistics by prevalence weighting, accomplished by an algorithmic automated iterative virtual population expansion. Cheng et al. (2020) op .cit.; and Cheng & Schmidt (2019) op .cit. The model was calibrated to the three month time point from ipilimumab and nivolumab monotherapy in immuno-oncology naïve melanoma trials, eventually including trials CA209038, CA209064, CA209067, and CA184169. The virtual population was calibrated to population mean, standard deviation, bins and distributions, with dozens of simultaneous fittings. Fisher's combined test provided a composite goodness of fit (a value between 0-1, the higher the better fit), and was checked to ensure the data were, overall, fit well by the VPop. Clinical data withheld from the calibration algorithm served for validation testing of the calibration.

To generate population level dose responses to non-fucosylated anti-CTLA-4 antibody therapy in anti-PD-1-progressed melanoma patients, we first simulated giving nivolumab 3 mg/kg Q2W for six doses to the virtual population. We then focused on the subpopulation of patients whose simulated index lesion size growth was bigger than 20%, based on the RECIST criteria, for the lesion scan at Day 84. The nivolumab progressed melanoma subpopulation gave the target population to test different non-fucosylated anti-CTLA-4 antibody doses and dose regimens on.

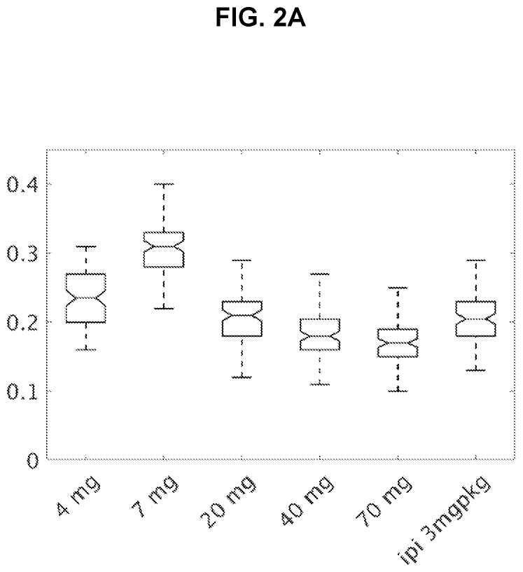

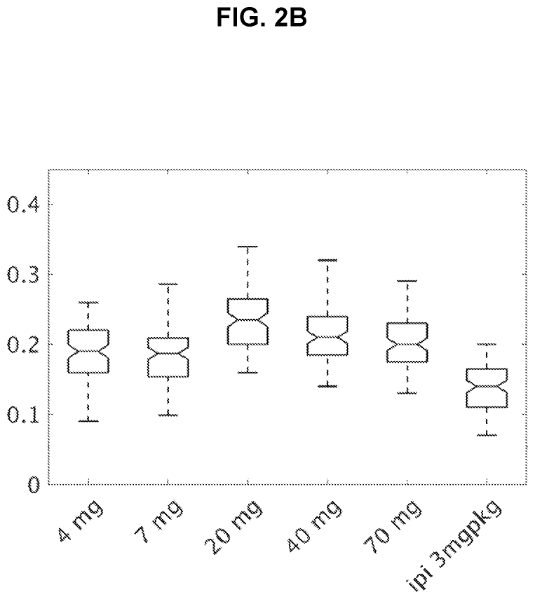

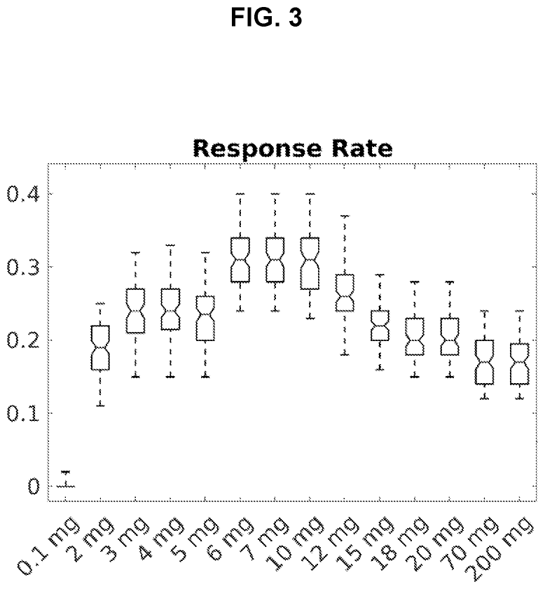

Results are provided at FIGS. 1A-1D, FIGS. 2A-2B and FIG. 3. FIGS. 1A-1D provide breakouts of the fraction of virtual patients into groups based on their index lesion response to treatment with non-fucosylated anti-CTLA-4 antibody determined using the first iteration of our QSP model. The second iteration of our QSP model was used to generate the data provided in FIGS. 2A and 2B, in which we tested a range of non-fucosylated anti-CTLA-4 antibody doses in these virtual patients, including 4 mg-70 mg given Q4W, and generated response predictions for the index lesions. Additional data from the second iteration QSP model are provided at FIG. 3. For each data set, variability between trials was modeled by generating data for 100 virtual trials, each comprising 100 virtual patients.

The model predicts a non-monotonic dose response for CTLA NF. It was projected that we would achieve optimal response rate at low doses, and the response rate would decrease as the dose was increased. The balance between CD8⁺ and CD4⁺ T_{reg} is crucial in determining tumor response. Given that non-fucosylated anti-CTLA-4 antibody can efficiently deplete both CD8⁺ and CD4⁺ T_{reg}, there is a clear mechanistic rationale for why model predicts non-monotonic dose responses based on the relative impact on the two cell populations. However, without a model-based approach, it is not obvious this would indeed happen and also would happen over a clinically accessible and important dose range. Without a model, the potential for not just decreased lesion responses but increasing progression also would not be as clear. The optimal dose varied with changes in the underlying physiology and time for washout of anti-PD-1 (e.g. two weeks versus six weeks), and in simulations the optimal dose was lower with higher response if non-fucosylated anti-CTLA-4 antibody was given earlier after the anti-PD-1 therapy was discontinued. Considered collectively, the QSP modeling results provided in the figures herein suggest that the optimal dose of non-fucosylated anti-CTLA-4 antibody for PD(L)1-progressed melanoma patients is between 4 mg and 10 mg, e.g. 5 mg to 7 mg, when administered two weeks after the last dose of nivolumab, or approximately 20 mg if administered six weeks after the last dose of nivolumab. The model predicts that a higher response rate might be achieved with a significantly lower dose level with enhanced ADCC capability when comparing to ipilimumab, at least in the case of PD(L)1-progressed melanoma patients.

### EXAMPLE 3

### Assay to Determine Percentage Non-Fucosylated Heavy Chains in a Sample of Anti-CTLA-4 Antibodies

Non-fucosylated anti-CTLA-4 mAb preparations are analyzed to determine the percentage of non-fucosylated heavy chains essentially as follows.

Antibodies are first denatured using urea and then reduced using DTT (dithiothreitol). Samples are then digested overnight at 37° C. with PNGase F to remove N-linked glycans. Released glycans are collected, filtered, dried, and derivatization with 2-aminobenzoic acid (2-AA) or 2-aminobenzamide (2-AB). The resulting labeled glycans are then resolved on a HILIC column and the eluted fractions are quantified by fluorescence and dried. The fractions are then treated with exoglycosidases, such as α(1-2,3,4,6) fucosidase (BKF), which releases core α(1,6)-linked fucose residues. Untreated samples and BKF-treated samples are then analyzed by liquid chromatography. Glycans comprising α(1,6)-linked fucose residues exhibit altered elution after BKF treatment, whereas non-fucosylated glycans are unchanged. The oligosaccharide composition is also confirmed by mass spectrometry. See, e.g., Zhu et al. (2014) MAbs 6:1474.

Percent nonfucosylation is calculated as one hundred times the molar ratio of (glycans lacking a fucose α1,6-linked to the first GlcNac residue at the N-linked glycan at N297 (EU numbering) of the antibody heavy chain) to (the total of all glycans at that location on all heavy chains (glycans lacking fucose and those having α1,6-linked fucose)).

TABLE 2
Summary of the Sequence Listing

| SEQ ID NO. | Description |
|---|---|
| 1 | human CTLA-4 (NP_005205.2) |
| 2 | human CD28 (NP_006130.1) |
| 3 | ipilimumab CDRH1 |
| 4 | ipilimumab CDRH2 |
| 5 | ipilimumab CDRH3 |
| 6 | ipilimumab CDRLI |
| 7 | ipilimumab CDRL2 |
| 8 | ipilimumab CDRL3 |
| 9 | ipilimumab heavy chain variable domain |
| 10 | ipilimumab light chain variable domain |
| 11 | ipilimumab heavy chain |
| 12 | ipilimumab heavy chain lacking C-terminal K |
| 13 | ipilimumab light chain |
| 14 | tremelimumab CDRH1 |
| 15 | tremelimumab CDRH2 |
| 16 | tremelimumab CDRH3 |
| 17 | tremelimumab CDRL1 |
| 18 | tremelimumab CDRL2 |
| 19 | tremelimumab CDRL3 |
| 20 | tremelimumab heavy chain variable domain |
| 21 | tremelimumab light chain variable domain |
| 22 | tremelimumab heavy chain |
| 23 | tremelimumab heavy chain lacking C-terminal K |
| 24 | tremelimumab light chain |

With regard to antibody sequences, the Sequence Listing provides the sequences of the mature variable regions and heavy and light chains, i.e. the sequences do not include signal peptides.

### Equivalents

Those skilled in the art will recognize, or be able to ascertain using no more than routine experimentation, many equivalents of the specific embodiments disclosed herein. Such equivalents are intended to be encompassed by the following claims.

1. A method of treating cancer in a human subject in need thereof comprising administering a non-fucosylated anti-CTLA-4 antibody as monotherapy at a dose of 4 mg, 5 mg, 6 mg, 7 mg, 10 mg, 20 mg, 40 mg, 70 mg, 100 mg or 200 mg at a dosing interval of once every two weeks (Q2W) or once every four weeks (Q4W).

2. The method of treating cancer of claim 1 wherein the dose is 5 mg, 7 mg, 10 mg, 20 mg, 40 mg or 70 mg.

3. The method of treating cancer of claim 1 wherein the dosing interval is Q2W.

4. The method of treating cancer of claim 1 wherein the non-fucosylated anti-CTLA-4 antibody comprises:
a. a CDRH1 consisting of the sequence of SEQ ID NO: 3;
b. a CDRH2 consisting of the sequence of SEQ ID NO: 4;
c. a CDRH3 consisting of the sequence of SEQ ID NO: 5;
d. a CDRL1 consisting of the sequence of SEQ ID NO: 6;
e. a CDRL2 consisting of the sequence of SEQ ID NO: 7; and
f. a CDRL3 consisting of the sequence of SEQ ID NO: 8.

5. The method of treating cancer of claim 4 wherein the non-fucosylated anti-CTLA-4 antibody comprises:
a. a heavy chain variable domain consisting of the sequence of SEQ ID NO: 9; and
b. a light chain variable domain consisting of the sequence of SEQ ID NO: 10.

6. The method of treating cancer of claim 5 wherein the non-fucosylated anti-CTLA-4 antibody comprises:
a. a heavy chain consisting of the sequence of SEQ ID NO: 11 or 12; and
b. a light chain consisting of the sequence of SEQ ID NO: 13.

7. The method of treating cancer of claim 1 wherein the non-fucosylated anti-CTLA-4 antibody comprises:
a. a CDRH1 consisting of the sequence of SEQ ID NO: 14;
b. a CDRH2 consisting of the sequence of SEQ ID NO: 15;
c. a CDRH3 consisting of the sequence of SEQ ID NO: 16;
d. a CDRL1 consisting of the sequence of SEQ ID NO: 17;
e. a CDRL2 consisting of the sequence of SEQ ID NO: 18; and
f. a CDRL3 consisting of the sequence of SEQ ID NO: 19.

8. The method of treating cancer of claim 7 wherein the antibody comprises:
a. a heavy chain variable domain consisting of the sequence of SEQ ID NO: 20; and
b. a light chain variable domain consisting of the sequence of SEQ ID NO: 21.

9. The method of treating cancer of claim 8 wherein the antibody comprises:
a. a heavy chain consisting of the sequence of SEQ ID NO: 22 or 23; and
b. a light chain consisting of the sequence of SEQ ID NO: 24.

10. The method of treating cancer of claim 1 wherein the cancer is selected from the group consisting of non-small cell lung cancer (NSCLC) (squamous and non-squamous), gastric cancer, triple-negative breast cancer (TNBC), colorectal cancer (CRC), squamous cell carcinoma of the head and neck (SCCHN), pancreatic cancer, metastatic castration resistant prostate cancer (mCRPC), and transitional cell cancer (urinary bladder) (TCC).

11. The method of treating cancer of claim 1 comprising administering the non-fucosylated anti-CTLA-4 antibody as monotherapy to a subject previously treated with an anti-PD-1 or an anti-PD-L1 antibody.

12. The method of treating cancer of claim 11 comprising administering the first dose of non-fucosylated anti-CTLA-4 antibody as monotherapy two to six weeks after the last dose of anti-PD-1 antibody or anti-PD-L1 antibody in the previous course of therapy.

13. The method of treating cancer of claim 12 comprising administering the first dose of non-fucosylated anti-CTLA-4 antibody as monotherapy two weeks after the last dose of anti-PD-1 antibody or anti-PD-L1 antibody in the previous course of therapy.

14. The method of claim 1 wherein the cancer is a cancer that is not eliminated, reduced or controlled by conventional treatment with nivolumab, and is selected from the group consisting of melanoma, non-small cell lung cancer (NSCLC) (squamous and non-squamous), gastric cancer, triple-negative breast cancer (TNBC), colorectal cancer (CRC), squamous cell carcinoma of the head and neck (SCCHN), pancreatic cancer, metastatic castration resistant prostate cancer (mCRPC), and transitional cell cancer (urinary bladder) (TCC).

15. A unit dose of a non-fucosylated anti-CTLA-4 antibody comprising:
a. 4 mg, 5 mg, 6 mg, 7 mg, 10 mg, 20 mg, 40 mg, 70 mg, 100 mg and 200 mg non-fucosylated anti-CTLA-4 antibody; and
b. one or more pharmaceutically acceptable excipients.

16. The unit dose of claim 15 comprising 5 mg, 7 mg, 10 mg, 20 mg, 40 mg or 70 mg non-fucosylated anti-CTLA-4 antibody.

17. The unit dose of claim 15 wherein the non-fucosylated anti-CTLA-4 antibody is non-fucosylated ipilimumab.

18. Use of a non-fucosylated anti-CTLA-4 antibody in the manufacture of a medicament for treating cancer at a fixed dose selected from the group consisting of 4 mg, 5 mg, 6 mg, 7 mg, 10 mg, 20 mg, 70 mg and 100 mg.

19. The use of claim 18 wherein the non-fucosylated anti-CTLA-4 antibody is non-fucosylated ipilimumab.
